(19) United States
(12) Patent Application Publication
(10) Pub. No.: US 2015/0335785 A1
(43) Pub. Date: Nov. 26, 2015

(54) CONSTRUCTS CONTAINING BONE TISSUE AND METHODS FOR MAKING THE SAME

(71) Applicant: Reed Ayers, Golden, CO (US)

(72) Inventor: Reed Ayers, Golden, CO (US)

(21) Appl. No.: 14/717,790

(22) Filed: May 20, 2015

Related U.S. Application Data
(60) Provisional application No. 62/000,964, filed on May 20, 2014.

(51) Int. Cl. A61L 27/36; A61L 27/04; A61L 27/12

(52) U.S. Cl. CPC A61L 27/3608; A61L 27/12; A61L 27/04; A61L 2430/40; A61L 2430/02

(57) ABSTRACT

The invention relates to a bone material scaffold or a synthetic bone material and a process for making the same. The bone material may comprise calcium phosphate, and the calcium phosphate may comprise multiple phases.

### CROSS REFERENCE TO RELATED APPLICATIONS

This application claims priority and the benefit under 35 U.S.C. §119(e) to U.S. Provisional Patent Application Ser. No. 62/000,964 filed May 20, 2014, which is incorporated herein in its entirety by reference.

### BACKGROUND

Serious body trauma caused by extensive battlefield injuries, such as that arising from high-velocity gunshot wounds, can lead to the loss of bone. In particular, battlefield activities can leave participants in need of having bones repaired by grafting. Autologous and cadaveric bone are considered the gold-standard bone graft materials. Their advantage is that they retain osteogenetic, osteoinductive, and osteoconductive properties that are required for bone regeneration. However, due to the nature of their harvesting, only a limited amount of bone tissue can be extracted. For autologous bone, detrimental side effects such as discomfort, donor site morbidity, secondary surgical procedures and risk of patient mortality or weakness resulting in fracture can occur. Allograft bone risks the transfer of antigens/disease, improper bone bonding, uncontrolled resorption and subsequent graft failure. These materials are often processed bone scaffolds. Bone particles are often only used to make putty.

There is significant demand for large scale, bioresorbable, biocompatible, and bioactive bone graft substitute materials (BGSM). Synthetic calcium phosphates represent an option for BGSM. Currently available synthetic calcium phosphate bone graft materials are limited to specific chemistries of calcium and phosphate due to the nature of their manufacturing processes, and by the available dimensions of materials. Two main methods are currently used to produce synthetic calcium phosphate bone graft materials. Some methods are wet methods, such as aqueous precipitation, gel casting, slurry dipping, spraying, sol-gel processes, or hydrolysis of calcium phosphates. A disadvantage of wet methods is that it can take weeks to produce small quantities of product. The second method utilizes solid-state reactions, which include uniaxial or isostatic compaction of loose powders, followed by a heat treatment. Other solid-state reactions include hot pressing, 3-dimensional laser printing and selective laser sintering. Solid-state reactions require high production time, cost and labor for bulk production and require multiple heat treatments to produce.

In addition to the need for bone graft materials, there is also a need for those bone graft materials to be resistant to microbial growth. Postoperative infections caused by gram positive bacteria (e.g. S. aureus, S. epidermidis, Streptococcus spp.) are one of the biggest challenges in battlefield orthopedic surgery. Incorporation of a localized antibiotic component, such as ionic sliver, within the implant could reduce the incidence of infection. Ionic silver is considered to have a broad spectrum of antimicrobial properties at concentrations as low as about 35 ppb without toxic effects to mammalian cells. It has been shown that silver (Ag) ions and Ag-based composites are highly toxic to microorganisms and incorporation of an antimicrobial component, for example silver based antimicrobial components, in the bone graft materials could create a localized antibiotic effect.

The present invention addresses these and other issues.

### SUMMARY

In the machining of bone tissue acquired in tissue banks, a significant amount of material is lost as dust or “powder” created from the cutting tools. This invention uses this scrap material as a feed source for a current aided diffusion (Spark Plasma Sintering [SPS] or Field Activated Synthesis [FAS]) wherein a current or electrical field is applied to a cold pressed construct of the bone powder to induce “welding” between the bone particles to create a new construct capable of being used in-vivo with minimal modification. The method may also be used on bone materials that have been milled for the purpose of becoming the precursor bone powder of the invention.

An aspect of the present invention is a method of producing a construct, for example, a bioactive BGSM, a graft, including a bone graft, or any other form of prosthetic that can be implanted into the body of a human or animal. The method includes providing a material to a cold press to form a pellet wherein the material comprises bone, and subjecting the material to a processing step to form the construct. The processing step includes sintering, or synthesis, or a combination of sintering and synthesis.

Another aspect of the invention is a method to form a construct. The method includes providing bone material to a calcium phosphate mold, then providing a current to the mold to form the construct.

Another aspect of the invention is a construct. The construct includes bone powder, and the construct is produced by a SPS process.

### BRIEF DESCRIPTION OF THE DRAWINGS

FIG. 1A illustrates an embodiment of a view of a set up of an apparatus using the SPS method; and

FIG. 1B illustrates an embodiment of a view of the set up of the apparatus 200 using the SPS method.

### DETAILED DESCRIPTION

The present invention includes a method for generating a construct product and its application in the orthopedic field. Furthermore, in some embodiments given the material of the construct itself is tissue, the resultant construct is not considered a medical device.

One skilled in the art would understand that the use of the final product may dictate or suggest the materials to use, and the concentrations of such materials in the manufacturing process. Calcium phosphate may contribute to the bioactivity, structure and mechanical properties of the final bone product. The bone particles may contribute to the bioactivity, the structure, and mechanical properties of the final bone product. Both materials are resorbable.

An aspect of the present invention is a method of manufacturing and using a non-equilibrium process to generate tissue constructs from tissue material currently considered scrap, as well as ground primary material. Another aspect of the present invention is a method of forming a construct using a SPS process. The process relied on the solid-state reaction at the interfaces between elemental particles in a green body. All of the materials remain solid throughout the manufacturing of the final product.

An aspect of the invention is a method to produce a construct using SPS. Raw bone powders are mixed with calcium phosphate then compacted (cold press) to create a green body or pellet. The pellet or green body is sintered, synthesized or simultaneously sintered and synthesized to produce a bone scaffold product.

The raw bone powders may be formed by machining, grinding, drilling, milling, cutting, boring, or the like, bone tissue. In some embodiments, the raw bone powder may be remnants from other bone processing methods. The material of the raw bone powders may be cancellous bone, cortical bone, allograft material, autograft material, xenograft material, or combinations thereof. The average size of the longest dimension of the particles of the raw bone powder may be between about 10 μm and about 1,000 μm. In some embodiments, the average side of the longest dimension of the particles of the raw bone powder may be about 10 μm, about 100 μm, about 200 μm, about 250 μm, about 300 μm, about 350 μm, about 400 μm, about 500 μm, about 600 μm, about 700 μm, about 800 μm, about 900 μm, or about 1000 μm. In some embodiments, the raw bone powder may be powder or dust.

The calcium phosphate may be tricalcium phosphate (TCP), hydroxyapatite (HA), or combinations thereof. In some embodiments, the tricalcium phosphate may be α-TCP, β-TCP, or combinations thereof.

In some embodiments, the calcium phosphate may include a dopant that had been previously incorporated into the calcium phosphate. Between about 0.005% by weight to about 30% by weight of the dopant in the calcium phosphate may be used, however, any suitable amount of the dopant can be added. The dopant may be an atom, an ion, a molecule, a compound or combinations thereof.

In some embodiments, the dopant may be an antimicrobial agent. The antimicrobial agent may be an atom, an ion, a molecule, or a compound. By way of non-limiting example, in some embodiments, the antimicrobial agent may be silver, gold, copper, zinc, silver nitrate or combinations thereof. In some embodiments, the antimicrobial agent may be silver (Ag) in the metallic or ion state. In some embodiments, the dopant can be strontium or SrO. Strontium may increase the compressive strength of the calcium phosphate material compared to TCP and HA scaffolds without strontium. In some embodiments, the dopant may be magnesium or MgO. Magnesium and strontium may increase bone formation, bioresorption and cellular activity of bone scaffolds. In some embodiments, HA and/or TCP may be added to the mixture in order to increase the concentration of HA and/or TCP in the final product or to control product properties.

Any suitable method may be used to mix the calcium phosphate and the bone powder. By way of example, a vibrational mixer may be used. Other suitable alternatives would be understood by one skilled in the art when considering the materials. Between about 0 weight percent to about 60 weight percent of the mixture may be calcium phosphate (with or without a dopant), with the remainder being the bone powder. In some embodiments, the between about 1 weight percent to about 60 weight percent of the mixture may be calcium phosphate. In some embodiments, the weight percent of calcium phosphate may be about 0 weight percent, about 1 weight percent, about 5 weight percent, about 10 weight percent, about 15 weight percent, about 20 weight percent, about 25 weight percent, about 30 weight percent, about 35 weight percent, about 40 weight percent, about 45 weight percent, about 50 weight percent, about 55 weight percent, or about 60 weight percent.

The sintering, synthesis, or combination of sintering and synthesis steps may occur in an oxygen environment, or in an oxygen depleted environment. In some embodiments, the steps may be performed in the presence of argon gas, nitrogen gas, helium gas, in a vacuum, and in a combination of gases or in a combination of gases with a vacuum. The pressure of the sintering, synthesis, or combination of sintering and synthesis steps may be between about 10 N to about 30 kN, in some embodiments about 200 kN. In some embodiments, the pressure may be about 10 N, about 500N, about 1kN, about 5 kN, about 10 kN, about 15 kN, about 20 kN, about 25 kN, or about 30 kN. A current applied during the sintering, synthesis, or combination of sintering and synthesis steps may be between about 1 A to about 100 A, in some embodiments about 50 A. In some embodiments, the current may be about 1 A, about 10 A, about 20 A, about 30 A, about 40 A, about 50 A, about 60 A, about 70 A, about 80 A, about 90 A, or about 100 A. The temperature during the sintering, synthesis, or combination of sintering and synthesis steps may be less than about 106° C. In some embodiments, the temperature may be between about 25° C. to about 100° C. In some embodiments, the temperature may be about 25° C., about 30° C., about 40° C., about 50° C., about 60° C., about 70° C., about 80° C., about 90° C., or about 100° C. A high voltage may be applied during the sintering, synthesis, or combination of sintering and synthesis steps. The voltage may be between about 1 V to about 200 V. In some embodiments, the voltage may be about 1 V, about 25 V, about 50 V, about 75 V, about 100 V, about 125 V, about 150 V, about 175 V, or about 200 V. The time duration of the sintering, synthesis, or combination of sintering and synthesis steps may be between about 10 seconds to about 300 seconds. In some embodiments, the time duration may be about 10 seconds, about 30 seconds, about 60 seconds, about 90 seconds, about 120 seconds, about 150 seconds, about 180 seconds, about 210 seconds, about 240 seconds, about 270 seconds, or about 300 seconds.

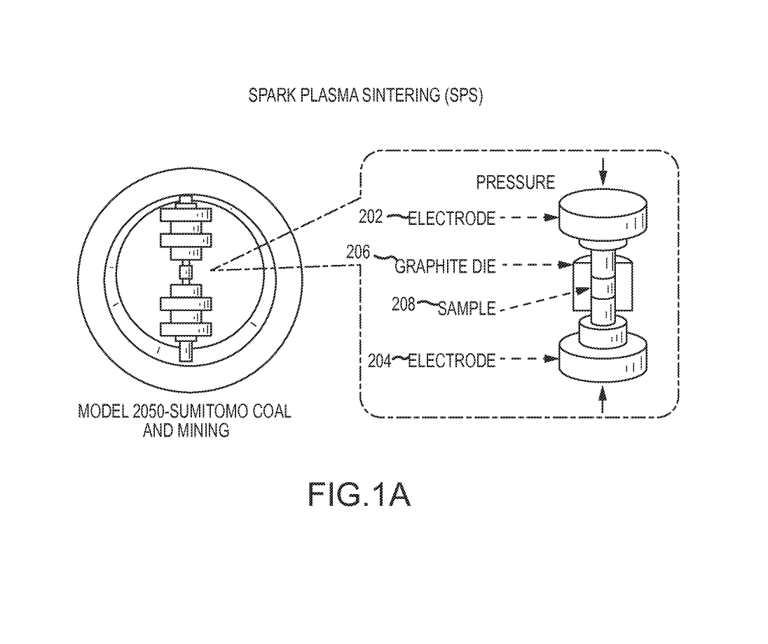

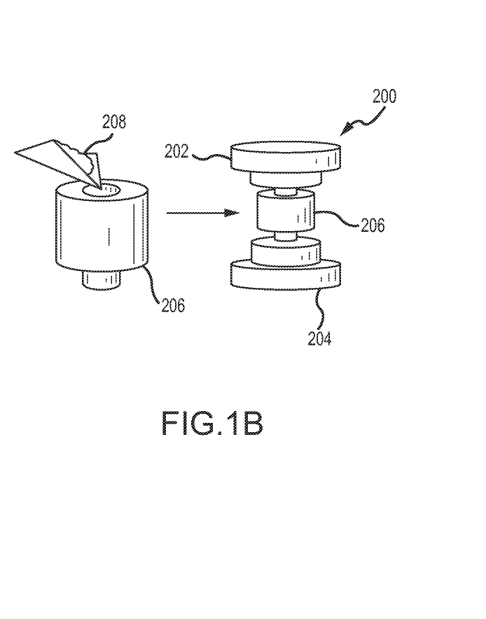

A mold may be used when the bone product is formed. The mold itself may act as a heating body to effect sintering via Joule heating and current aided diffusion (illustrated in FIG. 1A) or the mold can be removed and current is driven directly though the construct to effect Joule Heating between particles. The mold may be any suitable material. In some embodiments, the mold may be a dielectric material. Suitable dielectric materials include, but are not limited to, graphite. In some embodiments, the material of the mold may include, but is not limited to, steel, aluminum, titanium, or the like. In still other embodiments, the material of the mold may be made of a material that may be incorporated into the final product. By way of example, the mold may include magnesium, which may be incorporated into the bone product during the SPS process. In some embodiments, no mold is required to be used. By way of example, the mixed material may be pressed into the suitable shape then subjected to the sintering, synthesis, or combination of sintering and synthesis steps without the use of a mold.

The method may be used to produce a scaffold or body comprising entirely of bone powder, or may be a combination of the bone powder mixed with pre-existing calcium phosphate. The calcium phosphate may be in the form of powder, or may be a preexisting calcium phosphate scaffold. In some embodiments, a calcium phosphate scaffold may act as a mold in addition to a material to be joined to the bone particles in the final product. When the calcium phosphate is used as a mold, the bone particles may fill void spaces in the calcium phosphate. Current is then passed along the major axis for a sufficiently short time (micro-, milliseconds; possible seconds) sufficient to “melt” the collagen at particle interfaces to effect a joining of the particles. In some embodiments, the time period may be between about 10 seconds to about 300 seconds.

The bone product may be in the form of a pellet. The pellet formed may be any suitable shape or size. The shape and size of the pellet form may be chosen based on the intended application of the synthetic bone graft material. In some embodiments, the shape may be a cylinder, a cube, a sphere, an ovoid, a cuboid, an antiprism, a cupola, a hemisphere, a cone, a pyramid, a prism, a tube, a plate, or any other shape. One having skill in the art would understand that the dimensions of the pellet will depend upon the dimensions of the mold or the size capabilities of the device used to make the construct. Nevertheless, the dimensions of the construct may be any suitable dimension. By way of non-limiting example only, a diameter or width of a construct may be up to about 6 inches, or more. An advantage of the present invention is that it provides large bone product that may be cut to size for a particular use prior to entering a surgical room or in the surgical room. The parts may be machined using any suitable method, including but not limited to, machining and abrasion, and combinations thereof. In other embodiments, the mold may be used to produce a product suitable for use in the end product. By way of non-limiting example, a mold may be used to produce a product for use in spine surgery with little or no out further processing required to adjust the size of the product.

FIG. 1A illustrates an embodiment of a view of a set up of an apparatus using the SPS method. The apparatus includes electrodes 202 and 204, a graphite die 206, and the sample 208 within the die 206. Pressure may be applied to one or both ends of the electrode 202,204 to exert a pressure on the sample 208 to form a shaped material 208.

FIG. 1B illustrates an embodiment of a view of the set up of the apparatus 200 using the SPS method. The sample 208, which includes bone powder, and/or calcium phosphate, are added to the graphite die 206. The graphite die 206 is then placed into the apparatus 200.

The bone products made with the SPS process may be used in orthopedic applications. Bone powder can be used to create a construct of a size that is not dependent upon the size of the donor bone. Thus, larger constructs may be produced using the method of the invention. The constructs are capable of withstanding larger loads, and reduce fracturing potential. Furthermore, the bone products are not limited by the amount of bone provided since calcium phosphate may be used to produce a suitable sized product. Furthermore, multiple products may be produced from a large construct. Finally, the bone product uses bone powders efficiently. In some embodiments, the final product may be a scaffold.

Another aspect of the invention is a bone product. The bone product includes bone. In some embodiments, the bone product may include calcium phosphate. In some embodiments, the bone product may include a dopant. The dopant may be an atom, an ion, a molecule, a compound, or combinations thereof. In some embodiments, the dopant can be strontium or SrO. In some embodiments, the dopant may be magnesium or MgO. In still other embodiments, the dopant may be TCP or HA. By way of non-limiting example, in some embodiments, the dopant may be an antimicrobial agent such as silver, gold, copper, zinc, silver nitrate or combinations thereof. In some embodiments, the antimicrobial agent may be silver (Ag) in the metallic or ion state. The antimicrobial agent may be an atom, an ion, a molecule, or a compound. By way of non-limiting example, in some embodiments, the antimicrobial agent may be silver, gold, copper, zinc, silver nitrate or combinations thereof.

In some embodiments, where calcium phosphate is included in the bone product, the bone product can include HA, TCP, and combinations thereof. The TCP may be α-TCP, β-TCP or combinations thereof. The phase of calcium phosphate that may be in the bone product may affect the solubility and mechanical properties of the bone product. In some embodiments, between about 0% to about 100% by weight of the calcium phosphate in the bone product may be HA. In some embodiments, between about 0% to about 100% by weight of the calcium phosphate in the bone product may be 13-TCP. In some embodiments, between about 0% to about 100% by weight of the calcium phosphate in the bone product may be α-TCP.

The antimicrobial agent may be locally released into a patient near the implantation site. The rate of antimicrobial agent released from the implant may be a function of the infection. The antimicrobial agent may be released over a period between about hours to years after implantation. The duration of release and the release rate may be dependent upon the starting concentration of the antimicrobial agent in the product. The release rate may also be a function of the phase of calcium phosphate in the bone product as well as the porous structure of the bone product.

An additive material can be combined with the bone products. The additive material can be collagen, immunofluorescence label, alginate, chitosan coatings, BMPs, VEGF, or other proteins or mixtures thereof.

There are a variety of applications for the bone product. Multiple products are envisioned to be derived from this invention, including: orthopedic implants such as inter vertebral spacers, osteotomy wedges, bone scaffolds, and any production process calcium phosphates are needed. The bone product may be used as a large scale, bioresorbable, biocompatible, and bioactive BGSM for treatment of extensive battlefield injuries, accidental injuries, bone defects, craniofacial repair, dental applications as well as additional medical treatments that involve repairing or replacing bone materials that have been removed. The bone product may be suitable BGSM, wherein the fabrication and manufacturing of BGSM can potentially benefit from high purity, low cost materials that may be generated utilizing self-propagating reactions. The bone product may also be used to form scaffolds for tissue, that may be used, for example in ex-vivo reconstructions. In a specific embodiment, the biomimetic bone replacement material can be used for operations such as spinal fusion surgeries. The bone product can be used as a drug delivery device, as a carrier for growth factors, cells and/or proteins for bone tissue.

The foregoing description of the invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the invention. The embodiments described hereinabove are further intended to explain the best mode known for practicing the invention and to enable others skilled in the art to utilize the invention in such, or other, embodiments and with various modifications required by the particular applications or uses of the invention. It is intended that the appended claims be construed to include alternative embodiments to the extent permitted by the prior art.

1. A method of producing a construct, comprising:
providing a material to a cold press to form a pellet wherein the material comprises bone; and
subjecting the material to a process to form the construct, wherein the process is at least one of sintering, or synthesis.

2. The method of claim 1, wherein the material further comprises calcium phosphate.

3. The method of claim 2, wherein the calcium phosphate is at least one of a tricalcium phosphate, and a hydroxyapatite.

4. The method of claim 3, wherein the tricalcium phosphate is at least one of a α-tricalcium phosphate and a β-tricalcium phosphate.

5. The method of claim 2, wherein the calcium phosphate further comprises a dopant.

6. The method of claim 5, wherein the dopant is at least one of an atom, an ion, a molecule, or a compound.

7. The method of claim 2, wherein the material comprises between about 0 weight percent to about 60 weight percent of the calcium phosphate in the construct.

8. The method of claim 1, wherein the process comprises subjecting the material to a voltage of between about 1 V to about 200 V, a current between about 1 A to about 1000 A, a temperature between about 25° C. to about 100° C., and a pressure of between about 10 N to about 30 kN for a duration of between about 10 seconds to about 300 seconds.

9. The method of claim 5, wherein the dopant is at least one of a Mg, a Sr, a Sn, a silver, a gold, a copper, a zinc, and a silver nitrate.

10. A method to form a graft, comprising:
providing a mold, wherein the mold comprises calcium phosphate,
providing a bone material to the mold; and
providing an electrical current to the mold to form the graft.

11. The method of claim 10, wherein the bone material is a bone dust.

12. The method of claim 10, wherein the bone material is a bone powder.

13. The method of claim 10, further comprising mixing the bone material with a calcium phosphate material prior to providing the electrical current.

14. The method of claim 13, wherein the construct comprises between about 1-60 weight percent of the calcium phosphate.

15. The method of claim 10, wherein the calcium phosphate further comprising a dopant, wherein the dopant is at least one of a Mg, a Sr, a Sn, a silver, a gold, a copper, a zinc, and a silver nitrate.

16. The method of claim 1, wherein the calcium phosphate is at least one of a tricalcium phosphate, and a hydroxyapatite.

17. A bone graft comprising a bone powder, wherein the bone graft is produced by a SPS process, and wherein the bone graft is used in orthopedic procedures.

18. The construct of claim 17, wherein the construct further comprises calcium phosphate.

19. The construct of claim 18, wherein the calcium phosphate further comprising a dopant, wherein the dopant is at least one of a Mg, a Sr, a Sn, a silver, a gold, a copper, a zinc, and a silver nitrate, and combinations thereof.

20. The construct of claim 18, wherein the calcium phosphate is at least one of a tricalcium phosphate, and a hydroxyapatite.
